(19) United States
(12) Patent Application Publication
(10) Pub. No.: US 2010/0040693 A1
(43) Pub. Date: Feb. 18, 2010

(54) SILICA CAPSULES HAVING NANO-HOLES OR NANO-PORES ON THEIR SURFACES AND METHOD FOR PREPARING THE SAME

(75) Inventors: Bong Hyun Chung, Daejeon (KR); Yong Taik Lim, Daejeon (KR); Jin Kyeong Kim, Daejeon (KR)

Correspondence Address: MOORE & VAN ALLEN PLLC, P.O. BOX 13706, Research Triangle Park, NC, 27709, US

(73) Assignee: KOREA RESEARCH INSTITUTE OF BIOSCIENCE AND BIOTECH, Daejeon (KR)

(21) Appl. No.: 12/376,798

(22) PCT Filed: Aug. 1, 2007
(86) PCT No.: PCT/KR2007/003697

§ 371 Date: Jun. 26, 2009

(30) Foreign Application Priority Data
Aug. 9, 2006 (KR) 10-2006-0075104

(51) Int. Cl. A61K 9/14; B32B 5/16; H01F 1/00; G02F 1/361; A61K 38/43; A61K 38/02; C09K 11/06

(52) U.S. Cl. 424/489; 428/402; 252/62.51 R; 252/582; 424/94.1; 514/2; 252/301.16

(57) ABSTRACT

The present invention relates to silica capsules having nano-holes or nano-pores on the surface thereof, and preparation methods thereof. More specifically, relates to silica capsules having holes with a size ranging from a few nanometers (nm) to a few hundreds of nanometers (nm), on the surface thereof, multifunctional silica capsules containing magnetic nanoparticles and optical nanoparticles, and preparation methods thereof. According to the present invention, the silica capsules having holes on the surface thereof can be prepared by making an emulsion system using two fluids having different surface tensions, and selectively evaporating only one fluid of the two fluids during a process of forming a silica layer. In addition, the multifunctional silica capsules containing magnetic nanoparticles and optical nanoparticles can be prepared by loading various multifunctional nanoparticles into the two fluids.

### TECHNICAL FIELD

The present invention relates to silica capsules having nano-holes or nano-pores on the surface thereof, and preparation methods thereof, and more particularly to silica capsules having holes with a size ranging from a few nanometers (nm) to a few hundreds of nanometers (nm) on the surface thereof, multifunctional silica capsules containing magnetic nanoparticles and optical nanoparticles, and preparation methods thereof.

### BACKGROUND ART

Nano- and micro-sized silica particles have been actively used in various industrial fields, because they have advantages in that they are easy to prepare, and the surface thereof can be variously modified using silane chemistry. In particular, silica particles have very high biocompatibility, and thus have been actively applied in biological and medical fields. Particularly, in order to increase the stability of, for example, various organic dyes and enzymes, studies focused on loading such organic materials onto silica particles have been actively conducted, and studies on the use of silica particles as drug delivery carriers have also been actively conducted.

Meanwhile, hollow silica particles can contain a large amount of bioactive materials such as drugs therein, and thus have very excellent properties for use as delivery carriers. For this reason, many studies on the preparation of hollow silica capsules have been conducted (US 2005/0244322; U.S. Pat. No. 6,221,326; and WO 2004/006967). According to a method known to date, hollow silica capsules are prepared by coating a silica layer on polymer nanoparticles, and then melting the polymer layer. However, silica capsules prepared according to this method have limitations in that, because they have fine holes having a size of a few nanometers (nm), on the surface thereof, it is not easy to load drugs into the silica capsules, and particularly, proteins or drugs, having a size ranging from a few nanometers (nm) to a few tens of nanometers (nm) cannot be loaded into the silica capsules (Caruso, F et al, Advanced Materials, 13(14):1090, 2001; Van Bommel et al, Advanced Materials, 13(19):1472, 2001).

Also, current drug delivery systems have problems in that they serve only as drug carriers and it is impossible to guide drug delivery or molecular imaging of process.

For this reason, in the art, there is an urgent need to develop silica capsules, which have holes having a size ranging from a few nanometers (nm) to a few tens of nanometers (nm), on the surface thereof, and thus can efficiently carry bioactive materials such as drugs and, at the same time, enable drug delivery processes to be controlled and monitored.

Accordingly, the present inventors have made many efforts to develop silica capsules, which can efficiently carry bioactive materials and, at the same time, enable drug delivery processes to be controlled and monitored. As a result, the present inventors have found that silica capsules having holes on the surface thereof can be prepared by making an emulsion system using two fluids having different surface tensions, and selectively evaporating only one fluid of the two fluids during a process of forming a silica layer, and that multifunctional silica capsules having magnetic and optical properties can be prepared by loading various multifunctional nanoparticles into the two fluids, thereby completing the present invention.

### SUMMARY OF THE INVENTION

It is a main object of the present invention to provide a silica capsule holes with a size ranging from a few nanometers (nm) to a few hundreds of nanometers (nm), on the surface thereof, and a preparation method thereof.

Another object of the present invention is to provide a silica capsule having magnetic or optical properties, and a preparation method thereof.

Still another object of the present invention is to provide a material delivery system, comprising a biomolecule or a drug, loaded in said silica capsule.

To achieve the above objects, the present invention provides a silica capsule, which comprises a silica precursor and an amphiphilic substance and has holes or pores with a size of 1-1000 nm, formed on the surface thereof.

The present invention also provides a method for preparing a silica capsule having holes or pores with a size of 1-1000 nm formed on the surface thereof, the method comprising the steps of: (a) preparing an emulsion by dissolving an amphiphilic substance in distilled water, and then adding an organic solvent to the solution; (b) removing the organic solvent by heating the emulsion of step (a); and (c) adding a mixed solution of a basic material, a silica precursor and ethyl acetate to the resulting solution of step (b), and then allowing the mixture to stand.

The present invention also provides a silica capsule having magnetic or optical properties, which comprises a silica precursor and an amphiphilic substance, contains magnetic nanoparticles or optical nanoparticles inside or on the surface thereof, and has holes or pores with a size of 1-1000 nm, formed on the surface thereof.

The present invention also provides a method for preparing a silica capsule, which has holes or pores with a size of 1-1000 nm, formed on the surface thereof, and shows magnetic or optical properties, the method comprising the steps of: (a) preparing an emulsion by dissolving magnetic nanoparticles or optical nanoparticles and an amphiphilic substance in distilled water, and adding an organic solvent to the solution; (b) removing the organic solvent by heating the emulsion of step (a); and (c) adding a mixed solution of a basic material, a silica precursor and ethyl acetate to the resulting solution of step (b), and then allowing the mixture to stand.

The present invention also provides a system for delivering a material selected from the group consisting of biomolecules and drugs, the system comprising a biomolecule or drug loaded in a silica capsule, wherein the silica capsule comprises a silica precursor and an amphiphilic substance and has holes or pores with a size of 1-1000 nm, formed on the surface thereof, or the silica capsule shows magnetic or optical properties, comprises a silica precursor and an amphiphilic substance, contains magnetic nanoparticles or optical nanoparticles inside or on the surface thereof, and has holes or pores with a size of 1-1000 nm, formed on the surface thereof.

The present invention also provides a method for preparing a material delivery system, the method comprising loading a biomolecule or a drug into a silica capsule, wherein the silica capsule comprises a silica precursor and an amphiphilic substance and has holes or pores with a size of 1-1000 nm, formed on the surface thereof, or the silica capsule shows magnetic or optical properties, comprises a silica precursor and an amphiphilic substance, contains magnetic nanoparticles or optical nanoparticles inside or on the surface thereof, and has holes or pores with a size of 1-1000 nm, formed on the surface thereof.

Other features and aspects of the present invention will be apparent from the following detailed description and the appended claims.

### BRIEF DESCRIPTION OF DRAWINGS

FIG. 1 is a schematic diagram of a silica capsule, which contains magnetic nanoparticles and fluorescent nanoparticles and has holes on the surface thereof.

FIG. 2 is a SEM (scanning electron microscope) photograph of a silica capsule containing iron oxide nanoparticles.

FIG. 3 is a SEM photograph of a silica capsule containing CdSe/ZnS nanoparticles.

FIG. 4 is a SEM photograph of silica capsules, which have a structure shown in FIG. 1 and contain iron oxide nanoparticles and CdSe/ZnS nanoparticles.

FIG. 5 is a TEM (transmission electron microscope) photograph of a silica capsule, which have a structure shown in FIG. 1 and contains iron oxide nanoparticles and CdSe/ZnS nanoparticles (scale bar=200 nm).

FIG. 6 shows the magnetic properties and fluorescent properties of the silica capsules shown in FIG. 4 and FIG. 5.

FIG. 7 shows a fluorescent image of a nanoparticle-containing silica capsule, which exhibits fluorescent properties in the near-infrared region.

FIG. 8 shows cross-sectional photographs showing a polymer coated on the surface of a silica capsule prepared according to the present invention (scale bar=100 nm).

FIG. 9 shows confocal fluorescence microscopy images, taken after a silica capsule prepared according to the present invention was loaded with rhodamine dye.

### DETAILED DESCRIPTION OF THE INVENTION, AND PREFERRED EMBODIMENTS

In one aspect, the present invention relates to a silica capsule, which comprises a silica precursor and an amphiphilic substance and has holes or pores with a size of 1-1000 nm, formed on the surface thereof.

Also, the present invention relates to a method for preparing a silica capsule having holes with a size ranging from a few nanometers (nm) to a few hundreds of nanometers (nm), on the surface thereof, and to a method for preparing a silica capsule having not only magnetic properties, but also optical properties. As used herein, the term “silica capsule” refers to a silica particle having holes on the surface thereof, as described in Examples below. Also, as used herein, the term “amphiphilic substance” refers to a substance having a hydrophilic property and a hydrophobic property. Examples of the amphiphilic substance include C₁₀TAB (decyltrimethyl ammonium bromide), C₁₂TAB (dodecyltrimethyl ammonium bromide), C₁₄TAB (myristyltrimethyl ammonium bromide), C₁₆TAB (cetyltrimethyl ammonium bromide), C₁₈TAB (octadecyltrimethyl ammonium bromide) and C₁₆PC (ethylpyridinium chloride monohydrate).

In the present invention, the silica capsule is hollow and has holes with a size ranging from a few nanometers (nm) to a few hundreds of nanometers (nm), on the surface thereof and thus it is easy to load specific biomolecules or particles into the silica capsule. The prior silica capsules are hollow particles, whereas the inventive silica capsule is hollow and has holes on the surface thereof, and thus is advantageous in that it can efficiently carry bioactive materials such as drugs.

Also, the inventive silica capsule has an advantage in that it can be easily obtained through a one-step reaction. However, the prior silica capsules have problems in that the preparation process is complicated and also the efficiency thereof is low, because the prior silica capsules are prepared by coating a silica layer on polymer particles as templates to prepare silica particles, and then melting the polymer layer from the silica particles.

In the present invention, the inventive silica capsules were tested for magnetic properties and optical properties and, as a result, it could be observed that the silica capsules were attracted to a magnet and emitted light.

In the present invention, the silica precursor is preferably selected from the group consisting of TEOS (tetraethyl orthosilicate), TMOS (tetramethyl orthosilicate), APTES (aminopropyltriethoxysilane), APTMS (aminopropyltrimethoxysilane), MPTMS (3-mercaptopropyltrimethoxysilane) and MPTES (3-mercaptopropyltriethoxysilane), and the amphiphilic substance is preferably selected from the group consisting of C₁₀TAB (decyltrimethyl ammonium bromide), C₁₂TAB (dodecyltrimethyl ammonium bromide), C₁₄TAB (myristyltrimethyl ammonium bromide), C₁₆TAB (cetyltrimethyl ammonium bromide), C₁₈TAB (octadecyltrimethyl ammonium bromide) and C₁₆PC (ethylpyridinium chloride monohydrate), but the scope of the present invention is not limited thereto.

In another aspect, the present invention relates to a method for preparing a silica capsule having holes or pores with a size of 1-1000 nm formed on the surface thereof, the method comprising the steps of: (a) preparing an emulsion by dissolving an amphiphilic substance in distilled water, and then adding an organic solvent to the solution; (b) removing the organic solvent by heating the emulsion of step (a); and (c) adding a mixed solution of a basic material, a silica precursor and ethyl acetate to the resulting solution of step (b), and then allowing the mixture to stand.

The hollow structure of the inventive silica capsule having holes on the surface thereof is achieved by making an emulsion system using two fluids having different surface tensions, and evaporating one fluid of the two fluids in a process of forming a silica layer. Also, the size of the holes formed on the silica capsule can be controlled in the range of 1-1000 nm depending on reaction conditions.

In the present invention, the amphiphilic substance is preferably selected from the group consisting of C₁₀TAB (decyltrimethyl ammonium bromide), C₁₂TAB (dodecyltrimethyl ammonium bromide), C₁₄TAB (myristyltrimethyl ammonium bromide), C₁₆TAB (cetyltrimethyl ammonium bromide), C₁₈TAB (octadecyltrimethyl ammonium bromide) and C₁₆PC (ethylpyridinium chloride monohydrate), and the organic solvent is preferably selected from the group consisting of chloroform, dichloromethane, ethyl acetate-chloroform, dimethylformamide (DMF), N-methyl-2-pyrrolidone (NMP), dimethylacetamide (DMAc), cyclohexanone, ethyl alcohol, chlorobenzene, and ethyl ether, but the scope of the present invention is not limited thereto.

In the present invention, the basic material is preferably selected from the group consisting of NaOH, NH₄OH and KOH, and the silica precursor is preferably selected from the group consisting of TEOS (tetraethyl orthosilicate), TMOS (tetramethyl orthosilicate), APTES (aminopropyltriethoxysilane), APTMS (aminopropyltrimethoxysilane), MPTMS (3-mercaptopropyltrimethoxysilane) and MPTES (3-mercaptopropyltriethoxysilane), but the scope of the present invention is not limited thereto.

In the present method, the contents of the basic material, the silica precursor and the ethyl acetate in the mixed solution are preferably 2-5 vol %, 0.1-2 vol % and 0.1-7 vol %, respectively, based on the total solution volume.

In still another aspect, the present invention relates to a silica capsule having magnetic or optical properties, which comprises a silica precursor and an amphiphilic substance, contains magnetic nanoparticles or optical nanoparticles inside or on the surface thereof, and has holes or pores with a size of 1-1000 nm, formed on the surface thereof.

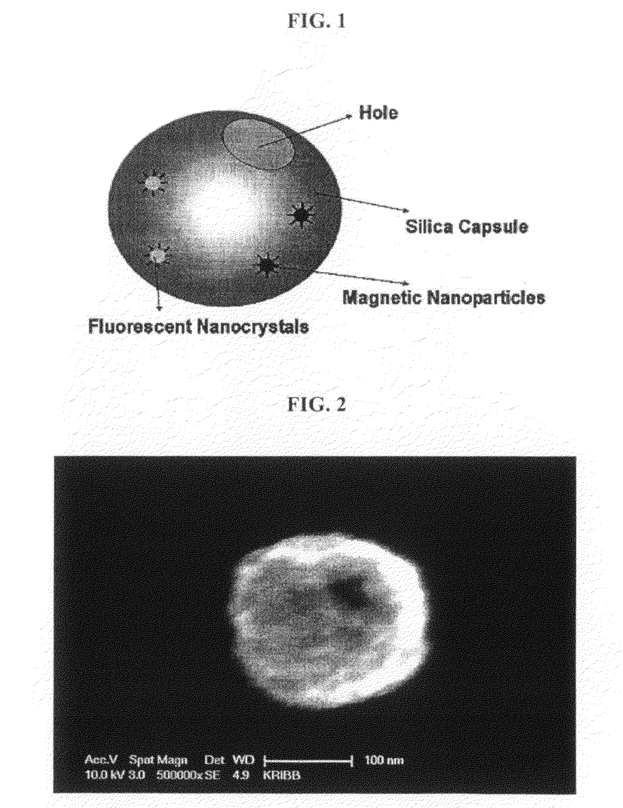

Also, multifunctional silica capsule having magnetic properties or optical properties can be prepared by adding functional nanoparticles having magnetic properties or optical properties during the process of preparing the silica capsule having holes on the surface thereof (see FIG. 1).

In the inventive silica capsule having magnetic properties or optical properties, the silica precursor is preferably selected from the group consisting of TEOS (tetraethyl orthosilicate), TMOS (tetramethyl orthosilicate), APTES (aminopropyltriethoxysilane), APTMS (aminopropyltrimethoxysilane), MPTMS (3-mercaptopropyltrimethoxysilane) and MPTES (3-mercaptopropyltriethoxysilane), and the amphiphilic substance is preferably selected from the group consisting of C₁₀TAB (decyltrimethyl ammonium bromide), C₁₂TAB (dodecyltrimethyl ammonium bromide), C₁₄TAB (myristyltrimethyl ammonium bromide), C₁₆TAB (cetyltrimethyl ammonium bromide), C₁₈TAB (octadecyltrimethyl ammonium bromide) and C₁₆PC (ethylpyridinium chloride monohydrate), but the scope of the present invention is not limited thereto.

In addition, the magnetic nanoparticles preferably comprise a material selected from the group consisting of Fe₂O₃, Fe₃O₄, FePt, Co and Gd (gadolinium), and the optical nanoparticles are preferably metal nanoparticles having a property of absorbing or scattering light and are selected from the group consisting of CdSe, CdSe/ZnS, CdTe/CdS, CdTe/CdTe, ZnSe/ZnS, ZnTe/ZnSe, PbSe, PbS InAs, InP, InGaP, InGaP/ZnS and HgTe, but the scope of the present invention is not limited thereto.

In still another aspect, the present invention relates to a method for preparing a silica capsule, which has holes or pores with a size of 1-1000 nm, formed on the surface thereof, and shows magnetic or optical properties, the method comprising the steps of: (a) preparing an emulsion by dissolving magnetic nanoparticles or optical nanoparticles and an amphiphilic substance in distilled water, and adding an organic solvent to the solution; (b) removing the organic solvent by heating the emulsion of step (a); and (c) adding a mixed solution of a basic material, a silica precursor and ethyl acetate to the resulting solution of step (b), and then allowing the mixture to stand.

In the present invention, the amphiphilic substance is preferably selected from the group consisting of C₁₀TAB (decyltrimethyl ammonium bromide), C₁₂TAB (dodecyltrimethyl ammonium bromide), C₁₄TAB (myristyltrimethyl ammonium bromide), C₁₆TAB (cetyltrimethyl ammonium bromide), C₁₈TAB (octadecyltrimethyl ammonium bromide) and C₁₆PC (ethylpyridinium chloride monohydrate), and the organic solvent is preferably selected from the group consisting of chloroform, dichloromethane, ethyl acetate-chloroform, dimethylformamide (DMF), N-methyl-2-pyrrolidone (NMP), dimethylacetamide (DMAc), cyclohexanone, ethyl alcohol, chlorobenzene, and ethyl ether, but the scope of the present invention is not limited thereto.

In the present invention, the basic material is preferably selected from the group consisting of NaOH, NH₄OH and KOH, and the silica precursor is preferably selected from the group consisting of TEOS (tetraethyl orthosilicate), TMOS (tetramethyl orthosilicate), APTES (aminopropyltriethoxysilane), APTMS (aminopropyltrimethoxysilane), MPTMS(3-mercaptopropyltrimethoxysilane) and MPTES (3-mercaptopropyltriethoxysilane), but the scope of the present invention is not limited thereto.

Also, in the present invention, the contents of the basic material, the silica precursor and the ethyl acetate in the mixed solution are preferably 2-5 vol %, 0.1-2 vol % and 3-7 vol %, respectively, based on the total solution volume.

In addition, the magnetic nanoparticles are preferably selected from the group consisting of Fe₂O₃, Fe₃O₄, FePt, Co and Gd (gadolinium), and the optical nanoparticles are preferably metal nanoparticles having a property of absorbing or scattering light and are selected from the group consisting of CdSe, CdSe/ZnS, CdTe/CdS, CdTe/CdTe, ZnSe/ZnS, ZnTe/ZnSe, PbSe, PbS InAs, InP, InGaP, InGaP/ZnS and HgTe, but the scope of the present invention is not limited thereto.

In still another aspect, the present invention relates to a system for delivering a material selected from the group consisting of biomolecules and drugs, the system comprising a biomolecule or drug loaded in said silica capsule.

In the present invention, the biomolecule or the drug is preferably selected from the group consisting of hormones, hormone analogues, enzymes, enzyme inhibitors, signal transduction proteins or their fragments, antibodies or their fragments, single-chain antibodies, binding proteins, binding domains, peptides, antigens, adhesion proteins, structural proteins, regulatory proteins, toxin proteins, cytokines, transcription regulatory factors, blood coagulation factors and plant defense-inducing proteins, but the scope of the present invention is not limited thereto.

In yet another aspect, the present invention relates to a method for preparing a material delivery system, the method comprising loading a biomolecule or a drug into said silica capsule.

The method for preparing a material delivery system preferably additionally comprises the steps of: (a) treating a material delivery system, having a biomolecule or drug loaded therein, with an anionic polyelectrolyte; (b) treating the anionic polyelectrolyte-treated material delivery system with a cationic polyelectrolyte; and (c) repeating the steps (a) and (b) 1-10 times to control the thickness of polymer shell.

The inventive method for preparing a material delivery system preferably comprises, before the step of loading the biomolecule or the drug, a step of treating the surface of the silica capsule with a molecule containing any one functional group selected from among carboxyl, thiol, biotin, streptavidin, aldehyde and amine groups. Also, the biomolecule or the drug is preferably selected from the group consisting of hormones, hormone analogues, enzymes, enzyme inhibitors, signal transduction proteins or their fragments, antibodies or their fragments, single-chain antibodies, binding proteins, binding domains, peptides, antigens, adhesion proteins, structural proteins, regulatory proteins, toxin proteins, cytokines, transcription regulatory factors, blood coagulation factors and plant defense-inducing proteins, but the scope of the present invention is not limited thereto.

In addition, the anionic polyelectrolyte is preferably PSS (poly(sodium 4-styrene-sulfonate), and the cationic polyelectrolyte is PAH (poly(allylamine hydrochloride) or PDADMAC (poly(diallyldimethylammonium chloride), but the scope of the present invention is not limited thereto.

### EXAMPLES

Hereinafter, the present invention will be described in further detail with reference to examples. It will be obvious to those skilled in the art that these examples are illustrative purpose only, and the scope of the present invention is not limited thereto.

### Example 1

### Preparation of Silica Capsules having Nano-holes on the Surface Thereof

0.1 g of CTAB (cetyltrimethyl ammonium bromide) (SIGMA, Germany) was dissolved in 5 ml of triple-distilled water, and the solution was rapidly stirred to make an emulsion. The emulsion was heated at 60° C. for 10 minutes to remove chloroform.

To 5 ml of the CTAB solution from which chloroform has been removed, 5 ml of triple-distilled water was added, and the mixture solution was stirred until it became uniform. Then, 0.3 ml of 2.5 mM NaOH, 0.05 ml of TEOS and 0.5 ml of ethylacetate were added thereto, and the mixture was stirred for 30 seconds and allowed to stand for 12 hours. Then, a process of centrifuging the solution at 5,000 rpm for 10 minutes and washing the centrifuged material with ethanol was repeated three times, thus preparing silica capsules having nano-holes on the surface thereof.

### Example 2

### Preparation of Silica Capsules Containing Magnetic Particles

7.5 mg of iron oxide nanoparticles were added to methanol (99.9%), and the solution was centrifuged at 4,000 rpm for 10 minutes and washed three times. Then, 7.5 mg of the pure iron oxide nanoparticles were dispersed in 5 mL of chloroform. Meanwhile, 0.1 g of CTAB (cetyltrimethyl ammonium bromide) (SIGMA, Germany) was dissolved in 5 ml of triple-distilled water. Then, the CTAB solution was mixed with the iron oxide nanoparticle solution, and the mixture solution was rapidly stirred to make an emulsion. The emulsion was heated at 60° C. for 30 minutes to remove chloroform.

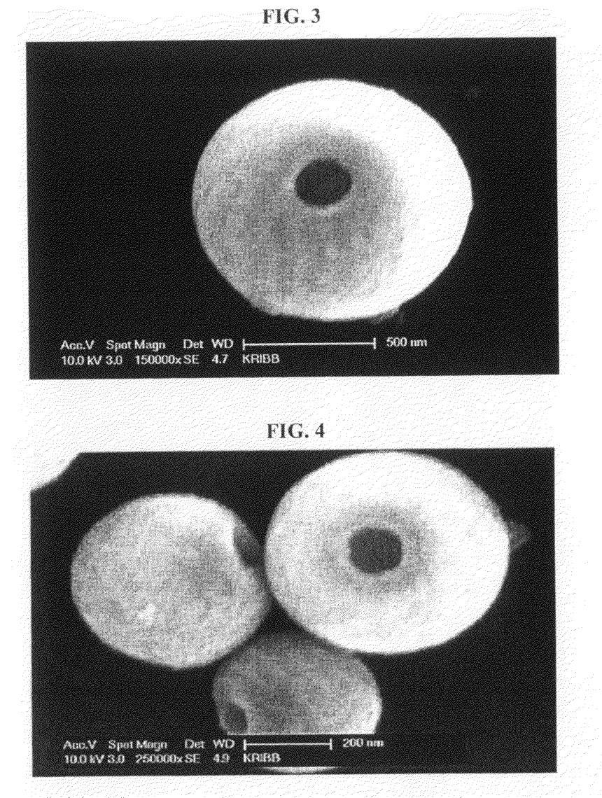

To 5 ml of the solution of iron oxide nanoparticle dispersed in CTAB, from which chloroform has been removed, 5 ml of triple-distilled water was added, and then the solution was stirred until it became uniform. Then, 0.3 ml of 2.5 mM NaOH, 0.05 ml of TEOS and 0.5 ml of ethylacetate were added thereto, and the mixture was stirred for 30 seconds and allowed to stand for 12 hours. Then, a process of centrifuging the solution at 5,000 rpm for 10 minutes and washing the centrifuged material with ethanol was repeated three times, thus preparing silica capsules containing magnetic nanoparticles (see FIG. 2). As a result, as it can be seen in FIG. 2, holes with a size of 50-100 nm existed on the surface of the silica capsules.

### Example 3

### Preparation of Silica Capsules Containing Optical Nanoparticles

7.5 mg of CdSe/ZnS nanoparticles (Evident Technologies, USA) were added to methanol (99.9%), and the solution was centrifuged at 4,000 rpm for 10 minutes and washed three times. Then, 7.5 mg of the pure CdSe/ZnS nanoparticles were dispersed in 5 ml of chloroform.

Meanwhile, 0.1 g of CTAB (cetyltrimethyl ammonium bromide) (SIGMA, Germany) was dissolved in 5 ml of triple-distilled water. Then, the CTAB solution was mixed with the CdSe/ZnS nanoparticle solution, and the mixture solution was rapidly stirred to make an emulsion. The emulsion was heated at 60° C. for 10 minutes to remove chloroform.

To 5 ml of the solution of CdSe/ZnS nanoparticle dispersed in CTAB, from which chloroform has been removed, 5 ml of triple-distilled water was added, and then the solution was stirred until it became uniform. Then, 0.3 ml of 2.5 mM NaOH, 0.05 ml of TEOS and 0.5 ml of ethylacetate were added thereto, and the mixture was stirred for 30 seconds and allowed to stand for 12 hours. Then, a process of centrifuging the solution at 5,000 rpm for 10 minutes and washing the centrifuged material with ethanol was repeated three times, thus preparing silica capsules containing fluorescent nanoparticles (see FIG. 3). As a result, as it can be seen in FIG. 3, holes with a size of 50-100 nm existed on the surface of the silica capsules.

### Example 4

### Preparation of Silica Capsules Containing Magnetic Nanoparticles and Optical Nanoparticles

7.5 mg of iron oxide nanoparticles were added to methanol (99.9%), and the solution was centrifuged at 4,000 rpm for 10 minutes and washed three times. Then, 7.5 mg of the pure iron oxide nanoparticles were dispersed in 5 mL of chloroform. Meanwhile, 0.1 g of CTAB (cetyltrimethyl ammonium bromide) (SIGMA, Germany) was dissolved in 5 ml of triple-distilled water. Then, the CTAB solution was mixed with the iron oxide nanoparticle solution, and the mixture solution was rapidly stirred to make an emulsion. The emulsion was heated at 60° C. for 10 minutes to remove chloroform, thus preparing solution 1.

Also, 7.5 mg of CdSe/ZnS nanoparticles (Evident Technologies, USA) were added to methanol (99.9%), and the solution was centrifuged at 4,000 rpm for 10 minutes and washed three times. Then, 7.5 mg of the pure CdSe/ZnS nanoparticles were dispersed in 5 ml of chloroform.

Meanwhile, 0.1 g of CTAB (cetyltrimethyl ammonium bromide) (SIGMA, Germany) was dissolved in 5 ml of triple-distilled water. Then, the CTAB solution was mixed with the CdSe/ZnS nanoparticle solution, and the mixture solution was rapidly stirred to make an emulsion. The emulsion was heated at 60° C. for 10 minutes to remove chloroform, thus preparing solution 2.

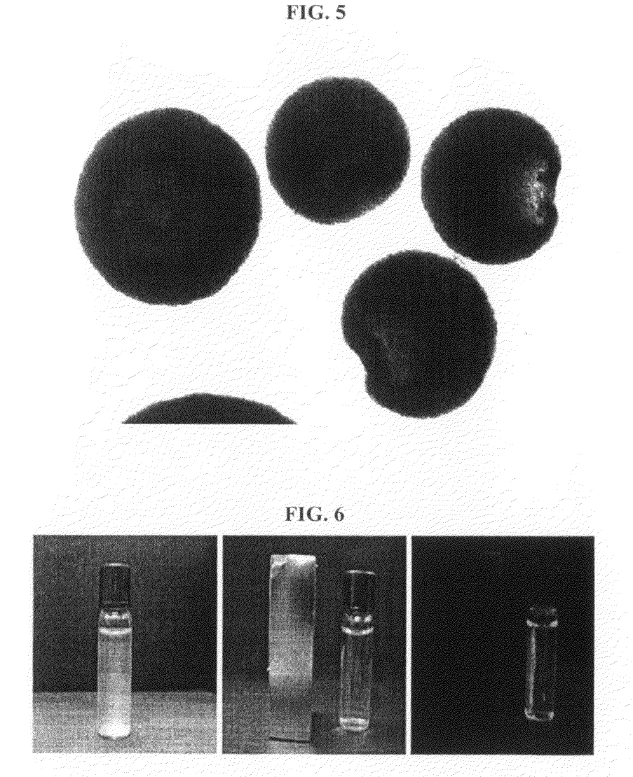

1 ml of the solution 1 and 1.5 ml of the solution 2 were mixed with each other, and the mixture solution was added to 7.5 ml of triple-distilled water and stirred until it became uniform. To the stirred solution, 0.3 ml of 2.5 mM NaOH, 0.05 ml of TEOS and 0.5 ml of ethylacetate were added, and the mixture was stirred for 30 seconds and allowed to stand for 12 hours. Then, a process of centrifuging the solution at 5,000 rpm for 10 minutes and washing the centrifuged material with ethanol was repeated three times, thus preparing silica capsules containing magnetic nanoparticles and fluorescent nanoparticles (see FIGS. 4 and 5). FIG. 4 is a SEM (scanning electron microscope) photograph of the inventive silica capsules containing iron oxide nanoparticles and CdSe/ZnS nanoparticles, and FIG. 5 is a TEM (transmission electron microscope) photograph of the silica capsules (scale bar=200 nm). As can be seen in FIGS. 4 and 5, holes with a size of 50-100 nm existed on the surface of the silica capsules.

### Example 5

### Magnetic Properties and Optical Properties of Silica Capsules

In order to examine the magnetic properties and optical properties of the inventive silica capsules, the magnetic properties and optical properties of the silica capsules prepared in Example 4 were examined (see FIG. 6). The above-prepared capsule sample was allowed to stand on a magnet (0.4 T, Nd-magnet, Magtopia, Korea) and, as a result, as shown in the photograph (middle) in FIG. 6, the silica capsules were attracted to the magnet. Also, the silica capsules were illuminated with 365-nm UV light and, as a result, as shown in the photograph (right) in FIG. 6, the silica capsules exhibited light. Thus, it could be seen that the inventive silica capsules had not only magnetic properties, but also optical properties.

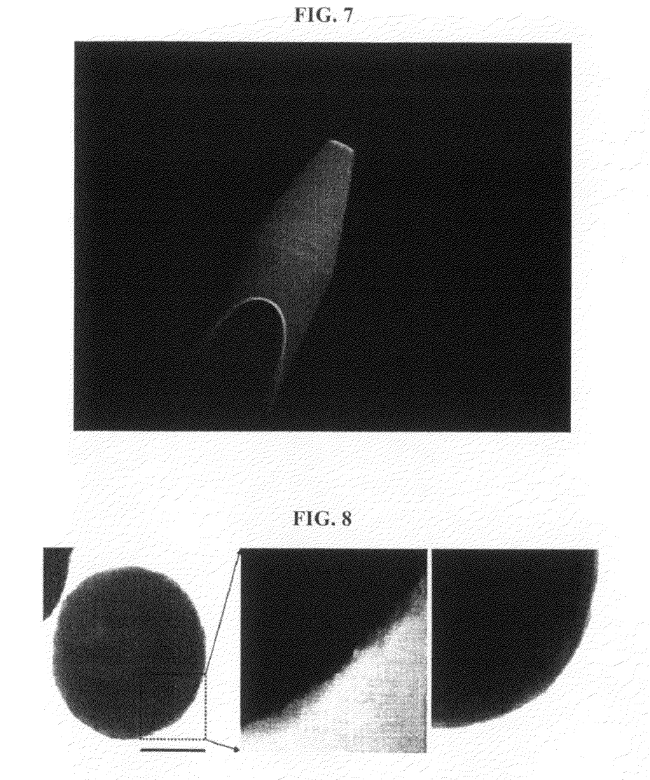

FIG. 7 is a fluorescent image of the silica capsules prepared in Example 4, taken in the near-infrared region. As can be seen in FIG. 7, the silica capsules of the present invention showed optical properties.

### Example 6

### Loading of Biomolecules and Nanoparticles into Silica Capsules

5 ml of a silica capsule solution prepared in each of Examples 1 to 4 was mixed with 1 ml of NH₂ solution, thus treating the surface of the silica capsules with the NH₂ solution. 1 ml of each of the surface-treated silica capsule solution was centrifuged at 5,000 rpm for 10 minutes and washed with triple-distilled water. Then, the silica capsule solution was dispersed in 1 ml of a solution obtained by dissolving 5 mg of CFP (cyanine fluorescent proteins), 5 mg of RFP (red fluorescent proteins), 1 mg of CdSe/ZnS (green fluorescent quantum dots), 1 mg of Rhodamin 6G and 1 mg of gold nanoparticles, and sonicated for 10 minutes. The sonicated solution was centrifuged at 5,000 rpm for 10 minutes. The supernatant was discarded and the precipitated silica capsules were collected.

The surface of the collected silica capsules had positive charges. For this reason, in order to neutralize the silica capsules, the precipitated silica capsules were dispersed in 1 ml of a solution obtained by dissolving negatively charged PSS (poly(sodium 4-styrene-sulfonate) in 0.5 M NaCl solution at a concentration of 1 mg/ml. Then, 20 μL of 0.1M HCl was added thereto, and the solution was sonicated for 10 minutes and centrifuged at 5,000 rpm for 10 minutes. The supernatant was discarded, and the precipitated silica capsules were washed three times with triple-distilled water (centrifugation at 5,000 rpm for 10 minutes after each washing), thus obtaining neutralized silica capsules.

In order to increase the thickness of the polymer shell, the obtained silica capsules were dispersed in 1 ml of a solution obtained by dissolving positively charged PDADMAC (poly(diallyldimethylammonium chloride) in 0.5 M NaCl at a concentration of 1 mg/ml, and the solution was sonicated for 10 minutes and centrifuged at 5,000 rpm for 10 minutes. The supernatant was discarded, and the precipitated silica capsules were washed three times with triple-distilled water (centrifugation at 500 rpm for 10 minutes after each washing). The PSS treatment process and the PDADMAC treatment process were repeated, thus obtaining silica capsules having increased polymer shell thickness. The obtained silica capsule solution was centrifuged at 5,000 rpm for 10 minutes, washed with triple-distilled water, and then dispersed in triple-distilled water for storage.

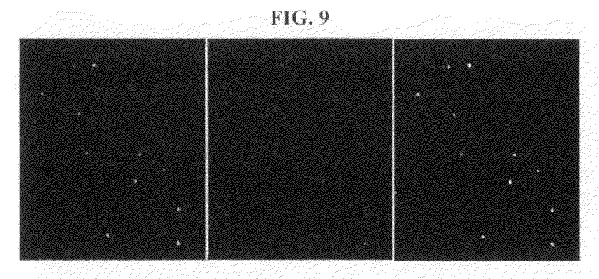

FIG. 8 is a TEM photograph of the biomolecule-loaded silica capsule (scale bar=100 nm), and FIG. 9 shows confocal fluorescence microscopy photographs taken after the prepared silica capsule was loaded with rhodamine dye. As can be seen in FIG. 8 and FIG. 9, the silica capsules of the present invention have biomolecules coated on the surface thereof.

### INDUSTRIAL APPLICABILITY

As described in detail above, the inventive silica capsules having holes on the surface thereof have advantages in that they can be used as carriers for delivering various materials such as biomolecules or drugs and enable the loading and delivery processes of such materials to be controlled. Also, the inventive silica capsules containing fluorescent nanoparticles and magnetic nanoparticles enable the above-mentioned material delivery processes to be monitored or guided using their optical properties and magnetic properties, and thus can be used in various fields, including biological and medial fields.

While the present invention has been described in detail with reference to specific features, it will be apparent to those skilled in the art that this description is only for a preferred embodiment and does not limit the scope of the present invention. Thus, the substantial scope of the present invention will be defined by the appended claims and equivalents thereof.

1. A silica capsule, which comprises a silica precursor and an amphiphilic substance and has holes or pores with a size of 1-1000 nm, formed on the surface thereof.

2. The silica capsule according to claim 1, wherein the silica precursor is selected from the group consisting of TEOS (tetraethyl orthosilicate), TMOS (tetramethyl orthosilicate), APTES (aminopropyltriethoxysilane), APTMS (aminopropyltrimethoxysilane), MPTMS (3-mercaptopropyltrimethoxysilane) and MPTES (3-mercaptopropyltriethoxysilane).

3. The silica capsule according to claim 1, wherein the amphiphilic substance is selected from the group consisting of C₁₀TAB (decyltrimethyl ammonium bromide), C₁₂TAB (dodecyltrimethyl ammonium bromide), C₁₄TAB (myristyltrimethyl ammonium bromide), C₁₆TAB (cetyltrimethyl ammonium bromide), C₁₈TAB (octadecyltrimethyl ammonium bromide) and C₁₆PC (ethylpyridinium chloride monohydrate).

4. A method for preparing a silica capsule having holes or pores with a size of 1-1000 nm, formed on the surface thereof, the method comprising steps of:
(a) preparing an emulsion by dissolving an amphiphilic substance in distilled water, and then adding an organic solvent to the solution;
(b) removing the organic solvent by heating the emulsion of step (a); and
(c) adding a mixed solution of a basic material, a silica precursor and ethyl acetate to the resulting solution of step (b), and then allowing the mixture to stand.

5. The method for preparing a silica capsule according to claim 4, wherein the amphiphilic substance is selected from the group consisting of C₁₀TAB (decyltrimethyl ammonium bromide), C₁₂TAB (dodecyltrimethyl ammonium bromide), C₁₄TAB (myristyltrimethyl ammonium bromide), C₁₆TAB (cetyltrimethyl ammonium bromide), C₁₈TAB (octadecyltrimethyl ammonium bromide) and C₁₆PC (ethylpyridinium chloride monohydrate).

6. The method for preparing a silica capsule according to claim 4, wherein the organic solvent is selected from the group consisting of chloroform, dichloromethane, ethyl acetate-chloroform, dimethylformamide (DMF), N-methyl-2-pyrrolidone (NMP), dimethylacetamide (DMAc), cyclohexanone, ethyl alcohol, chlorobenzene, and ethyl ether.

7. The method for preparing a silica capsule according to claim 4, wherein the basic material is selected from the group consisting of NaOH, NH₄OH and KOH.

8. The method for preparing a silica capsule according to claim 4, wherein the silica precursor is selected from the group consisting of TEOS (tetraethyl orthosilicate), TMOS (tetramethyl orthosilicate), APTES (aminopropyltriethoxysilane), APTMS (aminopropyltrimethoxysilane), MPTMS (3-mercaptopropyltrimethoxysilane) and MPTES (3-mercaptopropyltriethoxysilane).

9. The method for preparing a silica capsule according to claim 4, wherein the contents of the basic material, the silica precursor and the ethyl acetate in the mixed solution are 2-5 vol %, 0.1-2 vol % and 0.1-7 vol %, respectively, based on the total solution volume.

10. The silica capsule according to claim 1, comprising magnetic nanoparticles or optical nanoparticles inside or on the surface thereof.

11. (canceled)

12. (canceled)

13. The silica capsule having magnetic or optical properties according to claim 10, wherein the magnetic nanoparticles comprise a material selected from the group consisting of Fe₂O₃, Fe₃O₄, FePt, Co and Gd (gadolinium).

14. The silica capsule having magnetic or optical properties according to claim 10, wherein the optical nanoparticles are metal nanoparticles having a property of absorbing or scattering light.

15. The silica capsule having magnetic or optical properties according to claim 10, wherein the optical nanoparticles are selected from the group consisting of CdSe, CdSe/ZnS, CdTe/CdS, CdTe/CdTe, ZnSe/ZnS, ZnTe/ZnSe, PbSe, PbS InAs, InP, InGaP, InGaP/ZnS and HgTe.

16. The method for preparing a silica capsule according to claim 4, the method further comprising
dissolving magnetic nanoparticles or optical nanoparticles in the emulsion of step (a).

17.-21. (canceled)

22. The method for preparing a silica capsule according to claim 16, wherein the magnetic nanoparticles are selected from the group consisting of Fe₂O₃, Fe₃O₄, FePt, Co and Gd (gadolinium).

23. The method for preparing a silica capsule according to claim 16, wherein the optical nanoparticles are metal nanoparticles having a property of absorbing or scattering light.

24. The method for preparing a silica capsule according to claim 16, wherein the optical nanoparticles are selected from the group consisting of CdSe, CdSe/ZnS, CdTe/CdS, CdTe/CdTe, ZnSe/ZnS, ZnTe/ZnSe, PbSe, PbS InAs, InP, InGaP, InGaP/ZnS and HgTe.

25. The silica capsule according to claim 1, further comprising a biomolecule or drug loaded in the silica capsule.

26. The system for delivering a material according to claim 25, wherein the biomolecule or the drug is selected from the group consisting of hormones, hormone analogues, enzymes, enzyme inhibitors, signal transduction proteins or their fragments, antibodies or their fragments, single-chain antibodies, binding proteins, binding domains, peptides, antigens, adhesion proteins, structural proteins, regulatory proteins, toxin proteins, cytokines, transcription regulatory factors, blood coagulation factors and plant defense-inducing proteins.

27. A method for preparing a material delivery system, the method comprising loading a biomolecule or a drug into the silica capsule of claim 1.

28. The method for preparing a material delivery system according to claim 27, which additionally comprises the steps of:
(a) treating a material delivery system, having a biomolecule or drug loaded therein, with an anionic polyelectrolyte;
(b) treating the anionic polyelectrolyte-treated material delivery system with a cationic polyelectrolyte; and
(c) repeating the steps (a) and (b) 1-10 times to control the thickness of polymer shell.

29. The method for preparing a material delivery system according to claim 27, which comprises a step of treating the surface of the silica capsule with a molecule containing any one functional group selected from among carboxyl, thiol, biotin, streptavidin, aldehyde and amine groups, before the step of loading the biomolecule or the drug.

30. (canceled)

31. The method for preparing a material delivery system according to claim 27, wherein the anionic polyelectrolyte is PSS (poly(sodium 4-styrene-sulfonate), and the cationic polyelectrolyte is PAH (poly(allylamine hydrochloride) or PDADMAC (poly(diallyldimethylammonium chloride).
